(19) United States
(12) Patent Application Publication
(10) Pub. No.: US 2024/0383833 A1
(43) Pub. Date: Nov. 21, 2024

(54) Process for preparing an acetal from an olefin using an iodine-alkyl compound

(71) Applicant: Evonik Oxeno GmbH & Co. KG, Marl (DE)

(72) Inventors: Robert Franke, Marl (DE); Carolin Schneider, Monheim am Rhein (DE); Ralf Jackstell, Rostock (DE); Matthias Beller, Ostseebad Nienhagen (DE)

(73) Assignee: Evonik Oxeno GmbH & Co. KG, Marl (DE)

(21) Appl. No.: 18/649,671

(22) Filed: Apr. 29, 2024

(30) Foreign Application Priority Data
May 16, 2023 (EP) 23173655.4

(51) Int. Cl. C07C 41/54; B01J 31/22; B01J 31/24

(52) U.S. Cl. CPC C07C 41/54; B01J 31/2295; B01J 31/2457; B01J 2231/321; B01J 2531/004; B01J 2531/828

(57) ABSTRACT

Process for preparing an acetal from an olefin using an iodine-alkyl compound.

The present invention relates to a process for preparing an acetal from an olefin using an iodine-alkyl compound.

It was an object of the present invention to provide a process for preparing an acetal from an olefin. The intention here is to achieve a good yield.

This object is achieved by a process according to claim 1.

Process comprising the process steps of:
a) initially charging an olefin;
b) adding a compound of formula (I):

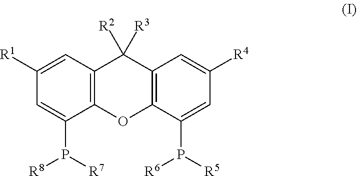

where R¹, R², R³, R⁴, R⁵, R⁶, R⁷, R⁸ are selected from: —H, —(C₁-C₁₂)-alkyl, —(C₆-C₂₀)-aryl;
and, if R¹, R², R³, R⁴, R⁵, R⁶, R⁷, R⁸ are —(C₆-C₂₀)-aryl, the aryl ring may have substituents selected from: —(C₁-C₁₂)-alkyl, —O—(C₁-C₁₂)-alkyl;
c) adding a Pt compound selected from: Pt(II)(COD)Me₂, diphenyl(1,5-COD)Pt(II), Pt(II)(acac)₂, Pt(0)(PPh₃)₄, Pt(0)(DVTS) solution (CAS: 68478-92-2), Pt(0)(ethylene)(PPh₃)₂, tris(benzylideneacetone)Pt(0), Pt(II)(OAC)₂ solution, Pt(0)(t-Bu)₂, Pt(II)(hexafluoroacetylacetonate)₂;
d) adding an iodine-alkyl compound;
e) adding an alcohol selected from: methanol, ethanol, 1-propanol, 1-butanol, 1-pentanol, 1-hexanol, 1-heptanol, 1-octanol, ethane-1,2-diol, propane-1,2-diol, propane-1,3-diol, butane-1,4-diol;
f) feeding in CO and H₂;
g) heating the reaction mixture from steps a) to f), to convert the olefin to an acetal.

In this process, process steps a) to f) can be effected in any desired sequence. Typically, however, CO and H₂ are added after the co-reactants have been initially charged in steps a) to e).

The expression (C₁-C₁₂)-alkyl encompasses straight-chain and branched alkyl groups having 1 to 12 carbon atoms. These are preferably (C₁-C₈)-alkyl groups, more preferably (C₁-C₆)-alkyl, most preferably (C₁-C₄)-alkyl.

Suitable (C₁-C₁₂)-alkyl groups are especially methyl, ethyl, propyl, isopropyl, n-butyl, isobutyl, sec-butyl, tert-butyl, n-pentyl, 2-pentyl, 2-methylbutyl, 3-methylbutyl, 1,2-dimethylpropyl, 1,1-dimethylpropyl, 2,2-dimethylpropyl, 1-ethylpropyl, n-hexyl, 2-hexyl, 2-methylpentyl, 3-methylpentyl, 4-methylpentyl, 1,1-dimethylbutyl, 1,2-dimethylbutyl, 2,2-dimethylbutyl, 1,3-dimethylbutyl, 2,3-dimethylbutyl, 3,3-dimethylbutyl, 1,1,2-trimethylpropyl, 1,2,2-trimethylpropyl, 1-ethylbutyl, 1-ethyl-2-methylpropyl, n-heptyl, 2-heptyl, 3-heptyl, 2-ethylpentyl, 1-propylbutyl, n-octyl, 2-ethylhexyl, 2-propylheptyl, nonyl, decyl.

The expression (C₆-C₂₀)-aryl encompasses mono- or polycyclic aromatic hydrocarbyl radicals having 6 to 20 carbon atoms. These are preferably (C₆-C₁₄)-aryl, more preferably (C₆-C₁₀)-aryl.

Suitable (C₆-C₂₀)-aryl groups are especially phenyl, naphthyl, indenyl, fluorenyl, anthracenyl, phenanthrenyl, naphthacenyl, chrysenyl, pyrenyl, coronenyl. Preferred (C₆-C₂₀)-aryl groups are phenyl, naphthyl and anthracenyl.

In one variant of the process, R², R³, R⁵, R⁶, R⁷, R⁸ are selected from: —(C₁-C₁₂)-alkyl, —(C₆-C₂₀)-aryl.

In one variant of the process, R⁵, R⁶, R⁷, R⁸ are —(C₆-C₂₀)-aryl.

In one variant of the process, R⁵, R⁶, R⁷, R⁸ are —Ph.

In one variant of the process, R² and R³ are —(C₁-C₁₂)-alkyl.

In one variant of the process, R² and R³ are —CH₃.

In one variant of the process, R¹ and R⁴ are —H.

In one variant of the process, the compound (I) has the structure (1):

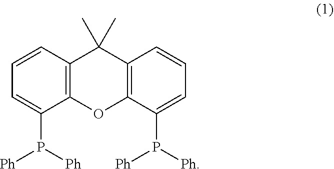 [SMILES: CC1(C)c2cccc(P(c3ccccc3)c3ccccc3)c2Oc2c(P(c3ccccc3)c3ccccc3)cccc21]

In one variant of the process, the Pt compound is Pt(II)(COD)Me₂.

In one variant of the process, the iodine-alkyl compound is selected from: ICH₂CH₂I, CH₃I, CH₂l₂, CHI₃, Cl₄, I₂CHCHI₂, I₃CCl₃, ICH₂CH₂CH₂I, ICH₂CH₂CH₂CH₂I, ICH₂CH₂CH₂CH₂CH₂I.

In one variant of the process, the iodine-alkyl compound is selected from: ICH₂CH₂l, CH₃I.

In one variant of the process, the iodine-alkyl compound is ICH₂CH₂I.

In one variant of the process, the iodine-alkyl compound is CH₃I.

In one variant of the process, the alcohol in process step e) is selected from: methanol, ethanol, 1-propanol, 1-butanol, ethane-1,2-diol, propane-1,2-diol.

In one variant of the process, the alcohol in process step e) is MeOH.

In one variant of the process, CO and H₂ are fed in at a pressure in a range from 1 MPa (10 bar) to 6 MPa (60 bar).

In one variant of the process, CO and H₂ are fed in at a pressure in a range from 3 MPa (30 bar) to 5 MPa (50 bar).

In one variant of the process, the reaction mixture is heated to a temperature in the range from 50° C. to 150° C.

In one variant of the process, the reaction mixture is heated to a temperature in the range from 70° C. to 130° C.

In one variant of the process, the olefin is selected from: ethene, propene, 1-butene, cis- and/or trans-2-butene, isobutene, 1,3-butadiene, 1,2-butadiene, 1-pentene, cis- and/or trans-2-pentene, 2-methyl-1-butene, 3-methyl-1-butene, 2-methyl-2-butene, hexene, tetramethylethylene, heptene, 1-octene, 2-octene, di-n-butene, tri-n-butene, 1,7-octadiene, 1,9-decadiene, methyl 9-decenoate (9-Dame) or mixtures thereof.

In one variant of the process, the olefin has two double bonds.

In one variant of the process, the olefin has two terminal double bonds.

In one variant of the process, this process comprises the additional process step f):
f) adding a solvent that is not an alcohol.

In one variant of the process, the solvent is selected from: THF, MTBE, DCM, ACN, heptane, DMF, toluene, xylene, mesitylene, dibenzyltoluene.

The invention is to be elucidated in detail hereinafter with reference to working examples.

### GENERAL EXPERIMENTAL DESCRIPTION

The olefin, an inert solvent, the alcohol, the Pt compound, the ligand and the halogen compound are placed in a stainless steel autoclave from Parr Instruments under argon. Synthesis gas CO/H₂ (1:1) is injected and the reaction is conducted at the chosen reaction temperature while stirring. After the end of the reaction time, the autoclave is cooled down to room temperature, the residual pressure is released, and a GC sample for determination of the yield of target product is taken and analysed.

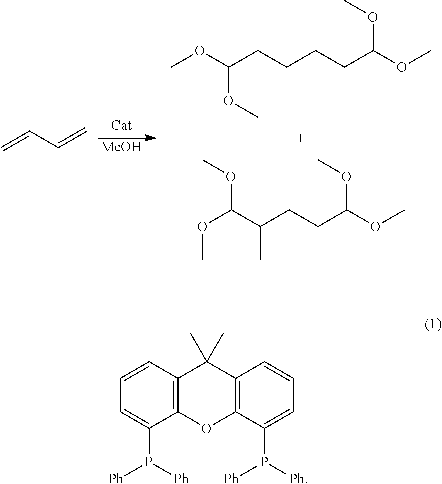

4.1 mmol of butadiene (3.2 ml of 8.3% stock solution in toluene), 80 mmol of MeOH (3.2 ml), 7 ml of toluene, 1.2 mol % of Pt(II)(COD)Me₂ (16.7 mg), 1.2 mol % of Xantphos (1) (29 mg) and 1.2 mol % of XCH₂CH₂X or 2.4 mol % of CH₃X are introduced into a 25 ml autoclave from Parr under argon. Synthesis gas CO: H₂=1:1 is injected to 40 bar and heated. The reaction is conducted while stirring. After the reaction has ended, the autoclave is cooled down, the pressure is released and the reaction solution is transferred into a Schlenk vessel. 10 mml of CH₂Cl₂ (0.6 ml) is added, and a ¹H NMR analysis and a GC analysis are carried out.

### Reaction Conditions

4.1 mmol butadiene, 1.2 mol % Pt(II)(COD)Me₂, 1.2 mol % Xantphos (1), solvent: toluene, alcohol: MeOH, p(CO/H₂): 40 bar (4 MPa).

These experimental results are listed in the table below.

### Variation of the Halogen

| Halogen compound | Temp [° C.] | t [h] | Yield [%] |
|---|---|---|---|
| BrCH₂CH₂Br* | 120 | 24 | <1 |
| ICH₂CH₂I | 120 | 16 | 36 |
| ICH₂CH₂I | 80 | 20 | 59 |

*noninventive comparative experiment

### Variation of the Iodine-Alkyl Compound

| Halogen compound | Temp [° C.] | t [h] | Yield [%] |
|---|---|---|---|
| CH₃I | 120 | 24 | 48 |
| ICH₂CH₂I | 120 | 16 | 36 |

As shown by the experimental results, the object is achieved by the process according to the invention.

1. Process comprising the process steps of:
a) initially charging an olefin;
b) adding a compound of formula (I): 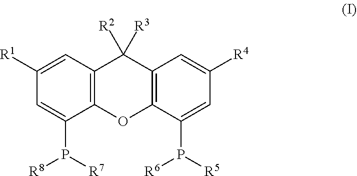
where R¹, R², R³, R⁴, R⁵, R⁶, R⁷, R⁸ are selected from: —H, —(C₁-C₁₂)-alkyl, —(C₆-C₂₀)-aryl; and, if R¹, R², R³, R⁴, R⁵, R⁶, R⁷, R⁸ are —(C₆-C₂₀)-aryl, the aryl ring may have substituents selected from: —(C₁-C₁₂)-alkyl, —O—(C₁-C₁₂)-alkyl;
c) adding a Pt compound selected from: Pt(II)(COD)Me₂, diphenyl(1,5-COD)Pt(II), Pt(II)(acac)₂, Pt(0)(PPh₃)₄, Pt(0)(DVTS) solution (CAS: 68478-92-2), Pt(0)(ethylene)(PPh₃)₂, tris(benzylideneacetone)Pt(0), Pt(II)(OAC)₂ solution, Pt(0)(t-Bu)₂, Pt(II)(hexafluoroacetylacetonate)₂;
d) adding an iodine-alkyl compound;
e) adding an alcohol selected from: methanol, ethanol, 1-propanol, 1-butanol, 1-pentanol, 1-hexanol, 1-heptanol, 1-octanol, ethane-1,2-diol, propane-1,2-diol, propane-1,3-diol, butane-1,4-diol;
f) feeding in CO and H₂;
g) heating the reaction mixture from steps a) to f), to convert the olefin to an acetal.

2. Process according to claim 1,
where R², R³, R⁵, R⁶, R⁷, R⁸ are selected from: —(C₁-C₁₂)-alkyl, —(C₆-C₂₀)-aryl.

3. Process according to claim 1, where R⁵, R⁶, R⁷, R⁸ are —(C₆-C₂₀)-aryl.

4. Process according to claim 1, where R² and R³ are —(C₁-C₁₂)-alkyl.

5. Process according to claim 1, where R′ and R⁴ are —H.

6. Process according to claim 1, wherein the compound (I) has the structure (1):

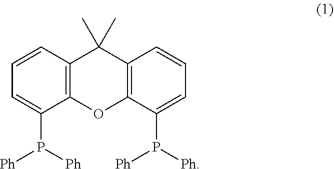 [SMILES: CC1(C)c2cccc(P(c3ccccc3)c3ccccc3)c2Oc2c(P(c3ccccc3)c3ccccc3)cccc21]

7. Process according to claim 1, wherein the Pt compound is Pt(II)(COD)Me₂.

8. Process according to claim 1, wherein the iodine-alkyl compound is selected from: ICH₂CH₂I, CH₃I, CH₂I₂, CHI₃, CI₄, I₂CHCHI₂, I₃CCI₃, ICH₂CH₂CH₂I, ICH₂CH₂CH₂CH₂I, ICH₂CH₂CH₂CH₂CH₂I.

9. Process according to claim 1, wherein the iodine-alkyl compound is selected from: ICH₂CH₂I, CH₃I.

10. Process according to claim 1, wherein the iodine-alkyl compound is ICH₂CH₂I.

11. Process according to claim 1, wherein the iodine-alkyl compound is CH₃I.

12. Process according to claim 1, wherein the alcohol in process step e) is selected from: methanol, ethanol, 1-propanol, 1-butanol, ethane-1,2-diol, propane-1,2-diol.

13. Process according to claim 1, wherein the alcohol in process step e) is MeOH.

14. Process according to claim 1, wherein the olefin is selected from: ethene, propene, 1-butene, cis-and/or trans-2-butene, isobutene, 1,3-butadiene, 1,2-butadiene, 1-pentene, cis-and/or trans-2-pentene, 2-methyl-1-butene, 3-methyl-1-butene, 2-methyl-2-butene, hexene, tetramethylethylene, heptene, 1-octene, 2-octene, di-n-butene, tri-n-butene, 1,7-octadiene, 1,9-decadiene, methyl 9-decenoate (9-Dame) or mixtures thereof.
